(19) United States
(12) Patent Application Publication
(10) Pub. No.: US 2024/0358969 A1
(43) Pub. Date: Oct. 31, 2024

(54) CATHETER INCLUDING AN ELONGATED BODY WITH INTERCONNECTING STRUTS

(71) Applicant: Covidien LP, Mansfield, MA (US)

(72) Inventors: Paul N. Jermihov, Irvine, CA (US); Daniel J. Deen, Long Beach, CA (US)

(21) Appl. No.: 18/647,821

(22) Filed: Apr. 26, 2024

Related U.S. Application Data
(60) Provisional application No. 63/498,680, filed on Apr. 27, 2023.

(51) Int. Cl. A61M 25/00

(52) U.S. Cl. CPC A61M 25/005; A61M 25/0045

(57) ABSTRACT

A medical device comprising: an elongated body extending along a longitudinal axis from a proximal end to a distal end, wherein the elongated body comprises a plurality of interconnecting struts extending around an outer perimeter of the elongated body and defining a plurality of openings, wherein the plurality of interconnecting struts comprises: a plurality of helical struts extending along the elongated body and defining one or more coils extending around the outer perimeter of the elongated body, a plurality of first cross struts extending between longitudinally adjacent coils of the one or more coils, and a plurality of second cross struts extending between first cross struts and coils of the plurality of helical struts, wherein each opening is defined by two or more struts, and an outer coating disposed on the outer surface of the elongated body.

### CROSS-REFERENCE TO RELATED APPLICATIONS

This application claims the benefit of and priority to U.S. Provisional Patent Application Ser. No. 63/498,680 filed Apr. 27, 2023, the entire disclosure of which is incorporated by reference herein.

### TECHNICAL FIELD

This disclosure relates to a medical catheter.

### BACKGROUND

A medical catheter defining at least one lumen has been proposed for use with various medical procedures. For example, in some cases, a medical catheter may be used to access and treat defects in blood vessels, such as, but not limited to, lesions or occlusions in blood vessels. A medical guidewire may be disposed within the catheter lumen and configured to control navigation of the medical catheter within the body of a patient.

### SUMMARY

A medical catheter, a medical guidewire, or other medical device may include an elongated body extending along a longitudinal axis. The elongated body may be configured to deflect away from the longitudinal axis to facilitate bending of the medical device as the medical device navigates within a patient (e.g., within vasculature of the patient) to a target location.

An outer coating may be disposed over the outer surface of the elongated body to inhibit unintended transfer of material into or from an inner lumen of the elongated body. The example elongated bodies of medical devices and systems described in this disclosure include a plurality of struts defining a plurality of openings on the outer surface of the elongated body. The plurality of openings may facilitate fixation of the outer coating to the elongated body. During fabrication of the medical device, a material forming the outer coating may be flowed into openings and around struts and subsequently solidified to become affixed to the struts along elongated body.

The plurality of struts may be arranged in one or more patterns that provide particular mechanical properties to the elongated body for improved navigation of the medical device through the vasculature. Some of the struts along the elongated body may include helical struts defining a helical shape extending along the longitudinal length. Coils of helical struts may be connected via a first set of cross struts and helical struts and the first set of cross struts may be connected via a second set of cross struts. Two or more of helical struts, struts of the first set of cross struts, and/or struts of the second set of cross struts may define openings along elongated body. This disclosure also describes examples of methods of forming the elongated bodies disclosed herein and methods of using medical devices having the example elongated bodies.

The example elongated bodies of medical devices described in the disclosure may provide several benefits over identical elongated bodies without openings and/or with different opening designs. The openings defined by the struts along elongated body may increase a fixation surface area between the material of the outer coating and the elongated body relative to identical elongated bodies without openings. The increased fixation surface area may increase the strength of the bond between the outer coating material and the elongated body and may reduce a likelihood of or inhibit separation of the outer coating from the outer surface of the elongated body compared to an identical elongated body without openings. The cross struts along the elongated body may improve mechanical properties (e.g., bending stiffness, tensile stiffness, torque responsiveness) of the elongated body compared to an identical elongated body without the cross struts. The methods for forming the openings in the elongated body as described in the disclosure may also increase the predictability of the performance of the elongated body and increase the applicability and utility of the elongated body as compared to other elongated bodies.

In some examples, this disclosure describes a medical device comprising: an elongated body extending along a longitudinal axis from a proximal end to a distal end, wherein the elongated body comprises: an inner surface defining an inner lumen extending along the longitudinal axis, and a plurality of interconnecting struts extending around an outer perimeter of the elongated body and defining a plurality of openings disposed on a portion of an outer surface of the elongated body and along the longitudinal axis and extending towards the inner lumen of the elongated body, wherein the plurality of interconnecting struts comprises: a plurality of helical struts, wherein each helical strut of the plurality of helical struts extends along the elongated body from the proximal end to the distal end and around the outer perimeter of the elongated body, and wherein the plurality of helical struts defines one or more coils extending around the outer perimeter along a longitudinal length of the elongated body, a plurality of first cross struts, wherein each first cross strut extends between longitudinally adjacent coils of the one or more coils, and a plurality of second cross struts, wherein each second cross strut extends between a first cross strut of the plurality of first cross struts and a coil of a helical strut of the plurality of helical struts, wherein each opening is defined by two or more of: one or more helical struts of the plurality of helical struts, one or more first cross struts of the plurality of first cross struts, or one or more second cross struts of the plurality of second cross struts; and an outer coating disposed on the outer surface of the elongated body, wherein at least a portion of the outer coating is disposed on sidewalls of the plurality of interconnecting struts.

In some examples, this disclosure describes a method of manufacturing a medical device, the method comprising: receiving, by processing circuitry of a device manufacturing system, a set of dimensions and locations of a plurality of openings disposed on an elongated body of the medical device based on mechanical properties of the medical device, wherein the elongated body comprises: an outer surface, an inner surface defining an inner lumen extending along a longitudinal axis, and a plurality of interconnecting struts extending around an outer perimeter of the elongated body and defining the plurality of openings extending from the outer surface towards the inner surface, wherein the plurality of interconnecting struts comprises: a plurality of helical struts, wherein each helical strut of the plurality of helical struts extends along the elongated body from the proximal end to the distal end and around the outer perimeter of the elongated body, and wherein the plurality of helical struts defines one or more coils extending around the outer perimeter along a longitudinal length of the elongated body, a plurality of first cross struts, wherein each first cross strut extends between longitudinally adjacent coils of the one or more coils, and a plurality of second cross struts, wherein each second cross strut extends between a first cross strut of the plurality of first cross struts and a coil of a helical strut of the plurality of helical struts, wherein each opening is defined by two or more of: one or more helical struts of the plurality of helical struts, one or more first cross struts of the plurality of first cross struts, or one or more second cross struts of the plurality of second cross struts; and causing, by the processing circuitry and based on the set of dimensions and locations of the plurality of openings, a cutting component of the device manufacturing system to form the plurality of openings on the outer surface of the portion of the elongated body.

In some examples, this disclosure describes a system comprising: a catheter comprising: an elongated body extending along a longitudinal axis from a proximal end to a distal end, wherein the elongated body comprises: an inner surface defining an inner lumen extending along the longitudinal axis, and a plurality of interconnecting struts extending around an outer perimeter of the elongated body and defining a plurality of openings disposed on a portion of an outer surface of the elongated body and along the longitudinal axis, wherein each opening of the plurality of openings extends from the outer surface towards the inner surface, wherein the plurality of interconnecting struts comprises: a plurality of helical struts, wherein each helical strut of the plurality of helical struts extends along the elongate body from the proximal end to the distal end and around an outer perimeter of the elongated body, and wherein the plurality of helical struts defines one or more coils extending around the outer perimeter along a longitudinal length of the elongated body, a plurality of first cross struts, wherein each first cross strut extends between longitudinally adjacent coils of the one or more coils, and a plurality second cross struts, wherein each second cross strut extends between a first cross strut of the plurality of first cross struts and a coil of a helical strut of the plurality of helical struts, wherein each opening is defined by two or more of: one or more helical struts of the plurality of helical struts, one or more first cross struts of the plurality of first cross struts, or one or more second cross struts of the plurality of second cross struts; and an outer coating disposed on the outer surface of the elongated body, wherein at least a portion of the outer coating is disposed on sidewalls of the plurality of struts; and a guidewire configured to be navigated within the inner lumen of the elongated body of the catheter.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example medical device with an elongated body extending along a longitudinal axis.

FIG. 2A is a conceptual diagram illustrating a distal portion of an example configuration of the elongated body of the example medical device of FIG. 1.

FIG. 2B is a conceptual diagram illustrating a proximal portion of an example configuration of the elongated body of the example medical device of FIG. 1.

FIG. 3 is a cross-section diagram illustrating a cross-section view of the elongated body of FIG. 2A, the cross-section being taken along line A-A in FIG. 2A.

FIG. 4 is a perspective diagram illustrating a side view of a portion of the elongated body of FIG. 1.

FIG. 5 is a conceptual diagram illustrating another side view of a portion of the elongated body of FIG. 1.

FIG. 6A is a graph illustrating an example bending stiffness of the elongated body of FIG. 1.

FIG. 6B is a graph illustrating an example torque responsiveness of the elongated body of FIG. 1.

FIG. 6C is a graph illustrating an example tensile stiffness of the elongated body of FIG. 1.

FIG. 7 is a flowchart illustrating an example process for forming the elongated body of FIG. 1.

FIG. 8 is a flowchart illustrating another example process for forming the elongated body of FIG. 1.

### DETAILED DESCRIPTION

In some examples, medical devices described herein include a medical catheter (“catheter”) that includes a relatively flexible elongated body configured to be navigated through vasculature of a patient, e.g., tortuous vasculature in a brain of the patient. The catheter may be navigated within the vasculature via use of a medical guidewire (“guidewire”). In other examples, medical devices described herein include the guidewire itself, which includes a relatively flexible elongated body. While the examples of this disclosure primarily describe the elongated body with reference to a catheter, an elongated body as described in any of the examples included herein may be used in other medical applications, e.g., as a part of a guidewire, as a part of another medical device configured to navigate within the body of the patient. The medical device includes a relatively flexible distal section that may exhibit increased flexibility relative to a proximal section of the medical device. The increased flexibility of the distal section may be at least partially (e.g., partially or fully) attributable to the configuration of the elongated body of the medical device.

The medical device includes an outer jacket (alternatively referred to herein as an “outer coating”) overlying the elongated body. During navigation, the outer jacket may contact walls of the vasculature through which the medical device is navigated, and the elongated body may bend in response to a change in direction of the vasculature. Such contact and/or bending may exert compressive, tensile, and/or shear forces on the outer coating, which may cause delamination of the outer coating from the elongated body.

The elongated body includes one or more openings which help to adhere the outer coating to the elongated body. The openings are defined by struts that define the elongated body. The openings may have any suitable configuration (e.g., any dimensions, any shapes) to increase fixation with the outer coating and/or to improve the mechanical properties of the elongated body compared to an elongated body that does not include openings. Each of the openings may be an absence of material in or a locally thinner portion of the wall of the elongated body (e.g., a groove, divot, pocket, through-hole, or the like in an otherwise continuous surface) that permits relative movement between the struts that define the respective opening. Each opening may extend partially from the outer surface of the elongated body towards an inner surface of the elongated body, or may extend entirely through the wall of the elongated body from the outer surface to the inner surface.

In addition to increasing adhesion of the outer jacket to the elongated body, the struts and openings are configured to provide various mechanical properties to the elongated body that aid in navigation of the medical device through vasculature. For example, the elongated body may require a minimum tensile stiffness and/or minimum tensile strength for a particular use of the medical device. Inadequate tensile stiffness or strength can translate into poor navigability of the medical device through vasculature of a patient when a clinician is advancing and retracting the medical device in tortuous anatomy. For example, if a distal tensile stiffness of the elongated body is inadequate, then the distal end of the medical device may not retract from the patient at the same rate as the proximal section, which may reduce the control over the medical device perceived by the clinician. In addition, if the tensile stiffness of the elongated body is inadequate, then the distal end of the medical device may remain in place while the proximal end stretches away from the distal end, which may reduce control over the medical device by the clinician. However, an increase in tensile stiffness, such as through inclusion of a stiffer material or a greater wall thickness, may correspondingly increase a bending stiffness, which may reduce navigability of the medical device by reducing flexibility of the elongated body compared to a medical device having an elongated body with a lesser tensile stiffness.

The elongated body includes struts and openings that are placed (i.e., positioned and/or oriented) and dimensioned to improve mechanical properties of the elongated body compared to an identical elongated body with different placement and/or dimensions of struts and/or openings. The plurality of openings and plurality of struts may provide for increased bending stiffness, tensile stiffness, tensile strength, or torque responsiveness compared to another elongated body with other opening designs and/or arrangements. The increased tensile stiffness and bending stiffness provided by the openings and struts in an elongated body described herein may translate into better navigability when a clinician is advancing or retracting the medical device, such as in tortuous anatomy. For example, the tensile strength of the elongated bodies defining one or more openings described herein may be sufficient to enable the distal section of the elongated body to be retracted from a patient without compromising the structural integrity of the medical device. Further, the tensile stiffness of the elongated bodies described herein may enable both the distal end and the proximal section of a medical device to be retracted from the vessel of a patient at a same or similar rate, which may provide the clinician with a perception of more control over the medical device.

The elongated body may include struts and openings arranged in various patterns, as described in further detail below. The one or more patterns may inhibit preferential bending of the elongated body and may increase torque responsiveness, tensile strength, and tensile stiffness of the elongated body. In some examples, the struts and/or openings are arranged in helical patterns along the longitudinal length of the elongated body. For example, helical struts of the plurality of struts may define a helical pattern along the longitudinal length of the elongated body, while interconnecting struts (also referred to herein as “cross struts”) may connect helical struts and/or other cross struts. Together, helical struts and/or cross struts may define the plurality of openings over the outer surface of the elongated body.

The elongated body may be configured to have different mechanical properties along the longitudinal axis of the elongated body. The elongated body may define a distal section and a proximal section, each of the distal and proximal section including a plurality of struts defining a plurality of openings. The dimensions of the openings and the struts may be uniform along the longitudinal length of the elongated body. In some examples, the dimensions of the openings and the struts at the distal section may be different from the dimensions of the openings and the struts at the proximal section. In such examples, the distal section of the elongated body may be more flexible than the proximal section of the elongated body.

By using the devices and techniques herein, the elongated body may have a sufficient tensile strength to allow a clinician to safely retract a distal section of the elongated body, even if, for example, the blood vessel around the distal end of the medical device is constricting the medical device. The tensile strength of the elongated body may help ensure that a distal section of the elongated body does not separate from a proximal the elongated body, e.g., during a medical procedure.

In some examples, the elongated body may accommodate delivery of various devices and/or fluids through the medical device. The elongated body may include a wall that defines an inner surface and an outer surface of the elongated body, such that the inner surface may define an inner lumen of the elongated body. In examples in which the one or more openings extend only partially through a thickness of a wall of an elongated body, the partial openings may be defined by an outer surface of the wall. By positioning the partial openings on an outer surface of the wall, an inner surface of the elongated body may remain substantially smooth (e.g., smooth or nearly smooth, or without projections or indentations that would inhibit the passage of a medical device), which may facilitate passage of one or more medical devices (e.g., guide members, an embolic protection device or an embolic retrieval device, and the like) through the inner lumen of the elongated body. Additionally, disposing the partial openings on the outer surface of the wall may facilitate disposal of a material forming the outer coating into the partial openings and affix the outer coating to the outer surface of the wall.

An elongated body with struts defining the openings may provide several benefits over an identical elongated body without struts or openings (e.g., a solid elongated body). The openings defined by the struts along elongated body may increase a fixation surface area between the material of the outer coating and the elongated body relative to the identical elongated body without the struts or openings. For example, the material of the outer coating with be disposed within openings and around at least some of the struts to increase the fixation surface area. The increased fixation surface area may increase the strength of the bond between the outer coating and the outer surface of the elongated body and may inhibit separation of the outer coating from the outer surface of the elongated body compared to an identical elongated body without openings.

The elongated body may be configured to exhibit a relatively high level of flexibility (e.g., bending stiffness), pushability (e.g., tensile stiffness), torqueability (e.g., torque responsiveness), and/or structural integrity (e.g., tensile strength). The elongated body may be configured to substantially conform to the curvature of the vasculature. In addition, in some examples, the elongated body may define a column strength and flexibility that allow at least a distal section of the medical device to be navigated from a femoral artery, through the aorta of the patient, and into the intracranial vascular system of the patient, e.g., to reach a relatively distal treatment site, including the middle cerebral artery (MCA), internal carotid artery (ICA), the Circle of Willis, and tissue sites more distal than the MCA, ICA, and the Circle of Willis. The MCA and, consequently, vasculature distal to the MCA may be relatively difficult to access due to the carotid siphon anatomy that must be traversed to reach such locations.

Although primarily described as being used to reach relatively distal vasculature sites, a medical device including an example elongated body described herein may readily be configured to be used with other target tissue sites. For example, the medical device may be used to access tissue sites throughout the coronary and peripheral vasculature, the gastrointestinal tract, the urethra, ureters, Fallopian tubes, and other body lumens.

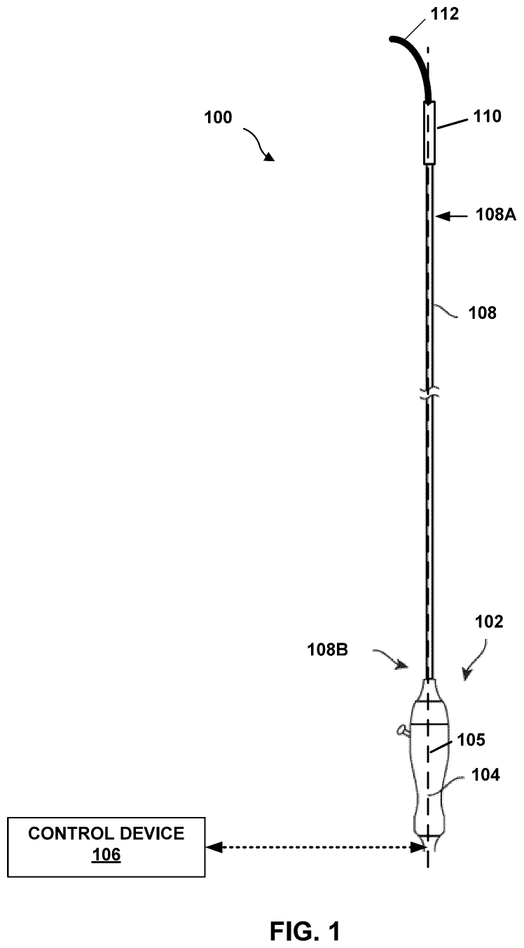

FIG. 1 is a conceptual diagram illustrating an example medical system 100 (also referred to herein as “system 100”) configured to be inserted into a patient. System 100 may include a medical device 102 including an elongated body 108 extending from a distal portion 108A to a proximal portion 108B along longitudinal axis 105 of device 102. A medical element 110 may be disposed along elongated body 108 (e.g., at a distal end of distal portion 108A of elongated body 108). A proximal portion 108B of elongated body 108 may be coupled to a handle 104 of device 102 and medical element 110 may be coupled to a control device 106 via one or more elements (e.g., electrodes, elongated tubes) disposed on or within elongated body 108. A clinician may navigate distal portion 108A of elongated body 108 within the patient via guide member 112. While FIG. 1 primarily describes the example elongated bodies described in this disclosure with respect to elongated body 108 of medical device 102, other example medical devices 102 or guide members 112 (e.g., a guide wire) may include a similar elongated body or an elongated body manufactured using the techniques described herein.

Elongated body 108 of device 102 may have any suitable outer diameter or longitudinal length. For example, a clinician may select device 102 with elongated body 108 having a specific outer diameter or longitudinal length based on an insertion location of device 102 on the patient, the target location within the patient, and/or the pathway between the insertion location and the target location. The outer diameter of elongated body 108 may be uniform or may vary along the longitudinal length of elongated body 108. In some examples, elongated body 108 is 2, 3, 4, 5, 6, or 7 French, or another suitable size. Elongated body 108 may be formed from a biocompatible material such as a biocompatible metallic alloy (e.g., nitinol) or a biocompatible polymer (e.g., PTFE). Elongated body 108 may define an elongated tube (e.g., a hypotube).

Elongated body 108 may include a plurality of struts along at least a portion of the longitudinal length of elongated body 108. The plurality of struts defines a plurality of openings along elongated body 108. Each opening may extend from the outer surface of elongated body 108 and towards an inner surface of elongated body 108 defining the inner lumen of elongated body 108. In some examples, openings may extend from the outer surface of elongated body 108 through to the inner lumen of elongated body 108. Openings may define different shapes and dimensions, e.g., due to different openings being defined by different combinations of struts. Openings may increase flexibility of elongated body 108 while the struts may increase torque responsiveness, tensile stiffness, and tensile strength of the elongated body 108, e.g., compared to an identical elongated body 108 without struts or with different openings. Openings may define helical patterns extending along the longitudinal length of elongated body 108 and around the outer perimeter of elongated body 108. The helical patterns of openings may inhibit preferential bending to elongated body 108 when elongated body 108 is within the patient.

An outer coating may be disposed over the outer surface of elongated body 108. The outer coating may facilitate movement of elongated body 108 within the patient. The outer coating may define a relatively smooth outer surface of device 102 relative to the outer surface of elongated body 108. For example, the outer coating may be disposed over elongated body 108 to prevent unintended interactions between tissue of the patient and the openings and struts of elongated body 108. The outer coating may isolate tissue of the patient from elongated body 108, e.g., to inhibit unintended introduction of foreign substances into the body of the patient.

A material of the outer coating may be disposed within openings and affixed to struts and/or around struts to increase adhesion of the outer coating to elongated body 108 compared to only disposing the material on the outer surface of elongated body 108. The openings and struts on elongated body 108 may increase a fixation area between the material of the outer coating and the material of elongated body 108, thereby increasing the strength of and/or a number of bonds between elongated body 108 and the outer coating. The increased bond strength and/or number of bonds may inhibit unintended separation of the outer coating from elongated body 108, e.g., in response to flexure of elongated body 108. A manufacturer may flow the material into openings and around struts and then solidify the material (e.g., via a heat shrink technique) to form the outer coating. The material may include, but is not limited to, a biocompatible polymer such as PEBA.

Medical element 110 may include an element configured to sense signals from the patient, an element configured to deliver a medical therapy to the patient, and/or an implantable medical device (IMD) configured to be implanted within the body of the patient. Medical element 110 may be coupled to control device 106 via elongated body 108 and may transmit signals and/or therapeutics between control device 106 and tissue of the patient.

Distal portion 108A of elongated body 108 is configured to be advanced within an anatomical lumen of the patient to locate medical element 110 at a target location within or otherwise proximate to the anatomical lumen. Medical element 110 may be disposed at distal portion 108A or at a distal end of elongated body 108. For example, elongated body 108 may be configured to position medical element 110 within a blood vessel, a ureter, a duct, an airway, or another naturally occurring lumen within the body of the patient.

The examples, described herein focus on the anatomical lumen being a blood vessel, but it will be understood that similar techniques may be used with other anatomical lumens. In certain examples, intravascular delivery of medical element 110 includes percutaneously inserting a guide member 112 into a vessel of the patient and moving elongated body 108 along guide member 112 until medical element 110 reaches a target location. Elongated body 108 may define an inner lumen for engaging guide element 112 (e.g., a guidewire) for delivery of medical element 110 using over-the-wire (OTW) or rapid exchange (RX) techniques. In some examples, device 102 may be a steerable or non-steerable device configured for use without guide member 112.

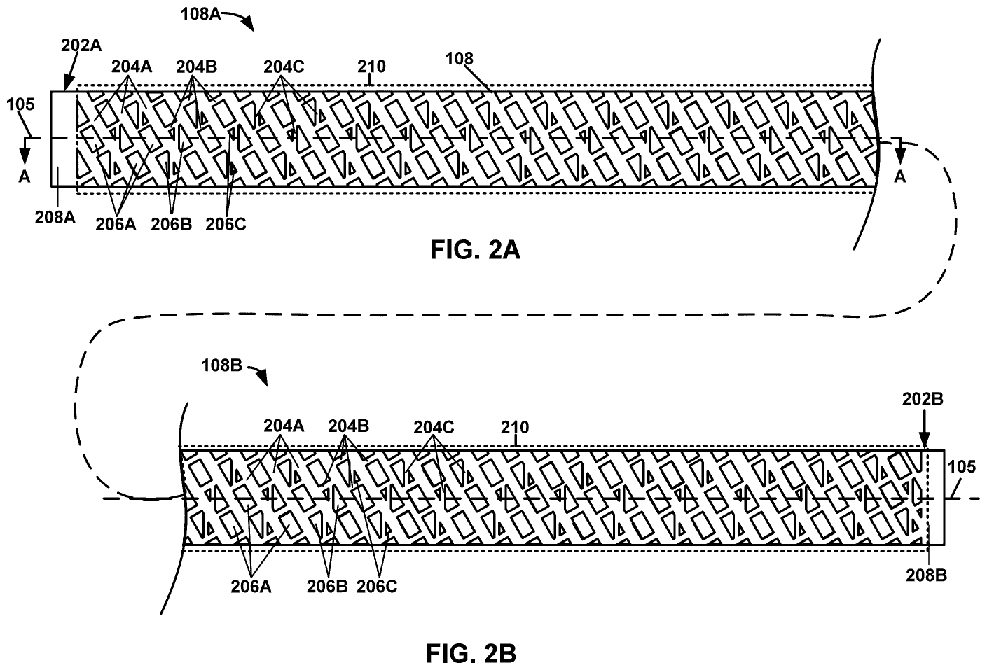

As mentioned above, medical devices described herein include elongated bodies that include struts and openings defined by the struts that are configured to provide improved mechanical properties and increased adhesion compared to medical devices that do not include, or that include differently configured, struts and openings. FIG. 2A is a conceptual diagram illustrating distal portion 108A of an example configuration of elongated body 108 of the example medical device 102 of FIG. 1, while FIG. 2B is a conceptual diagram illustrating proximal portion 108B of an example configuration of elongated body 108 of the example medical device 102 of FIG. 1.

Elongated body 108 may extend along longitudinal axis 105 from a distal end 202A to a proximal end 202B. Elongated body 108 includes a plurality of struts 204A-C (collectively referred to herein as “struts 204”) defining a plurality of openings 206A-C (collectively referred to herein as “openings 206”). An outer coating 210 of device 102 may be disposed over an outer surface of elongated body 108, within openings 206, along sidewalls of struts 204, and/or around struts 204. Outer coating 210 may define an outer surface of device 102 (e.g., radially outward from longitudinal axis 105).

Distal portion 108A of elongated body 108 may define a distal section 208A encompassing distal end 202A, while proximal portion 108B of elongated body 108 may define a proximal section 208B encompassing proximal end 202B. Distal and proximal sections 208A, 208B (collectively referred to herein as “end sections 208”) may not include any struts 204 and openings 206, e.g., to improve integrity of elongated body 108. In such examples, as illustrated in FIGS. 2A and 2B, struts 204 and openings 206 may extend from a proximal end of distal section 208A to a distal end of proximal section 208B. In some examples, end sections 208 include struts 204 and openings 206. In such examples, struts 204 and openings 206 extend from distal end 202A to proximal end 202B.

Struts 204 are configured to provide various mechanical properties to elongated body 108 that improve navigability of the medical device. To provide particular mechanical properties, elongated body 108 includes struts 204 having different dimensions, positions, and orientations. In the example of FIGS. 2A and 2B, struts 204 include, but are not limited to, a plurality of helical struts 204A, a plurality of first cross struts 204B, and a plurality of second cross struts 204C. Helical struts 204A may extend around the outer perimeter of elongated body 108 and along the longitudinal length of elongated body 108. Helical struts 204A may extend along the entire length of elongated body 108, e.g., from proximal end 202B or proximal section 208B to distal end 202A or distal section 208A. Each helical strut 204A may define one or more helical coils along the length of elongated body 108. The pitch of the one or more coils (i.e., a longitudinal distance between longitudinal adjacent coils) may be uniform or may vary along the length of elongated body 108. The one or more coils may define a helix or spiral having a corresponding helical angle. The helical angle may be uniform or may vary along the length of elongated body 108. Each helical strut 204A may be parallel to one or more other helical struts 204A. Helical struts 204A may rotate about longitudinal axis 105 in a clockwise or counterclockwise direction. Elongated body 108 may include one or more helical struts 204A (e.g., two, three, four, five, or six or more helical struts 204A).

First cross struts 204B may extend between longitudinally adjacent helical coils of helical struts 204A. The longitudinally adjacent helical coils may be parts of a single helical strut 204A or may be parts of different helical struts 204A. For each first cross strut 204B, a first end may be connected to a first coil and a second end may be connected to a second coil. Each first cross strut 204B may extend linearly from the first end to the second end or may define a curvature (e.g., an arc, a wave pattern) from the first end to the second end. In some examples, first cross strut 204B may extend from the first coil to a third coil that is not longitudinally adjacent to the first coil (e.g., a coil that is longitudinally separated from the first coil by one or more coils).

Second cross struts 204C may extend between a helical strut 204A and a first cross strut 204B. Second cross strut 204C may intersect helical strut 204A and/or first cross strut 204B along the length of helical strut 204A or first cross strut 204B or at an intersection between helical strut 204A and first cross strut 204B. Second cross strut 204C may extend linearly along the outer surface of elongated body 104 from helical strut 204A to first cross strut 204B or may define a curvature between helical strut 204A and first cross strut 204B.

Struts 204 define a plurality of openings 206. Openings 206 may permit relative movement of struts 204. Openings 206 may have uniform or varying shapes and dimensions. Openings 206 may define triangular, rectangular, square, quadrilateral, trapezoidal, pentagonal, or hexagonal shapes. For example, as illustrated in FIGS. 2A and 2B, openings 206A may define rectangular shapes and openings 206B, 206C may define triangular shapes of different dimensions. Each opening 206 may be formed by two or more struts 204A, e.g., by one or more helical struts 204A, one or more first cross struts 204B, and/or one or more second cross struts 204C.

In some examples, openings 206 bordering end sections 208 are defined by two struts 204, e.g., by one helical strut 204A and one first cross strut 204B. Each opening 206A may be defined by two helical struts 204A and two first cross struts 204B. Openings 206A may define helical patterns extending around the outer perimeter of elongated body 108 and along the longitudinal length of elongated body 108. Each of openings 206B, 206C may be defined by one helical strut 204A, one first cross strut 204B, and one second cross strut 204C. In some examples, second cross struts 204C may extend through some of openings 206A to define a pair of openings 206B, 206C. Each pair of openings 206B, 206C may be separated by a second cross strut 204C.

Outer coating 210 may define an outer surface of device 102 and may be disposed over elongated body 108. A material forming outer coating 210 may be disposed within openings 206, attached to sidewalls some of struts 204, and/or be disposed around some of struts 204. In some examples, a manufacturer flows the material into opening 206 and/or around struts 204 and at a temperature of up to about 300 degrees Celsius (° C.) to about 400° C. A heat shrink material may be applied over the material of the outer coating 210 to control flow of the material within openings 206 and struts 204. The heat shrink material may be removed to expose outer coating 210 after the material of the outer coating 210 becomes affixed to elongated body 108. Outer coating 210 may define a relatively smooth outer surface of device 102, e.g., to facilitate navigation of device 102 within a body lumen of the patient.

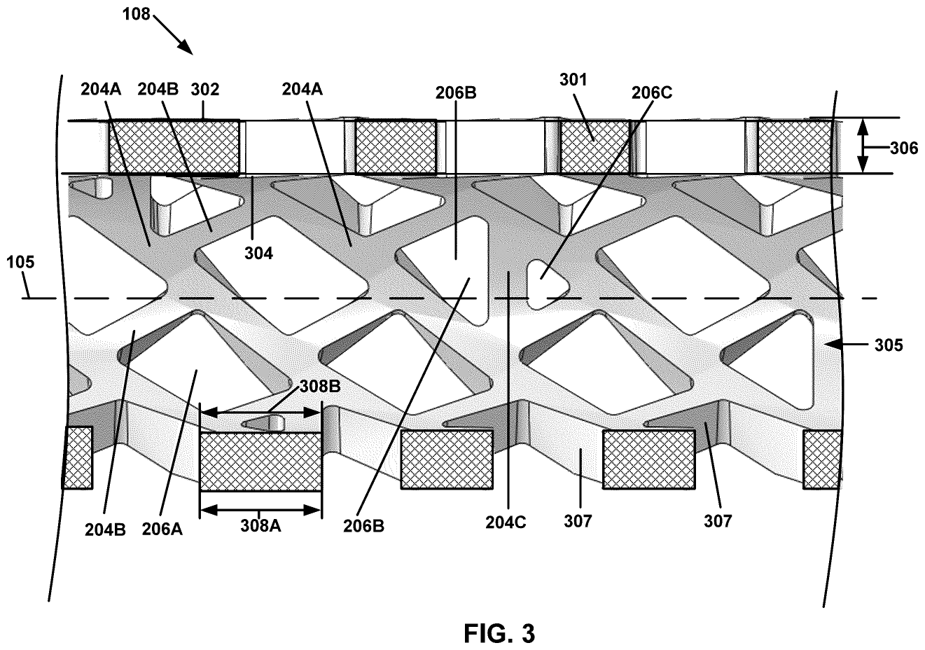

FIG. 3 is a cross-section diagram illustrating a cross-section view of the elongated body 108 of FIG. 2A, the cross-section being taken along line A-A in FIG. 2A. As illustrated in FIG. 3, openings 206 may extend through wall 301 of elongated body 108 from outer surface 302 of elongated body 108 to inner surface 304 of elongated body 108. Inner surface 304 may define an inner lumen 305 of elongated body 108. Inner lumen 305 may be configured to receive guide member 112, e.g., as a part of navigation of device 102 within the patient.

Wall 301 may be defined by struts 204. Struts 204 may extend from outer surface 302 to inner surface 304. Each strut 204 may include a first surface along outer surface 302, a second surface along inner surface 304, and two sidewalls 307 extending along the length of strut 204 and connecting the first surface to the second surface. Each strut 204 may define sidewalls 307 with corresponding thicknesses 306 from outer surface 302 towards inner surface 304 of elongated body 108 along a reference plane orthogonal to longitudinal axis 105.

Each strut 204 may define a depth 306 (or thickness). Depth 306 may be less than or equal to the thickness of wall 301 from outer surface 302 to inner surface 304. Depth 306 may be uniform or may vary along the length of elongated body 108. In some examples, sidewalls 307 of struts 204 at distal portion 108A of elongated body 108 may define depths 306 less than sidewalls 307 of struts 204 at proximal portion 108B of elongated body 108, e.g., to increase the flexibility of distal portion 108A relative to proximal portion 108B. Openings 206 may define a depth from outer surface 302 towards inner surface 304. In some examples where openings 206 extend from outer surface 302 to inner surface 304, as illustrated in FIG. 3, the depth of openings 206 is equal to depth 306 of struts 204 defining openings 206.

Each strut 204 may define a width. The width of each strut 204 may be measured along a reference plane orthogonal to strut 204. Struts 204 may define an outer width 308A at outer surface 302 of elongated body 108 and an inner width 308B at inner surface 304 of elongated body 108. A manufacturer may form struts having different widths 308A, 308B, e.g., to adjust bending stiffness, tensile stiffness, tensile strength, or torque responsiveness of elongated body 108. In some examples, the manufacturer may use a laser-cutting implement or another cutting implement to remove material from elongated body 108 to form openings 204 and struts 206B. In such examples, struts 206 may define an inner width 308B less than or equal to outer width 308A, e.g., due to the shape of the elongated body 108 and the position of the laser-cutting implement or another cutting implement relative to elongated body 108. Different types of struts 204 may define different widths. For example, helical struts 204A, first cross struts 204B, and second cross struts 204C may same, similar, or different widths. The width of each strut 204 may be uniform or may vary along the length of the strut 204.

In some examples, as illustrated in FIG. 3, each strut 204 may define a rectangular cross-section. In some examples, depending on widths 308A, 308B of struts 204, some struts 204 define a quadrilateral or trapezoidal cross-section. Struts 204 may define quadrilateral cross-sections to facilitate extension of struts 204 around the outer perimeter of elongated body 108 and to facilitate rotation of struts 204 about longitudinal axis 105. As illustrated in FIG. 3, each opening 206 may include a plurality of corners. Corners of openings 206 may be rounded or beveled to increase contact between sidewalls of struts 204 and the material of outer coating 210. Rounding or beveling corners may also decrease stress on the material of outer coating 210 at the corners and may inhibit unintended separation of the material of outer coating 210 from elongated body 108 (e.g., from sidewalls of struts 204).

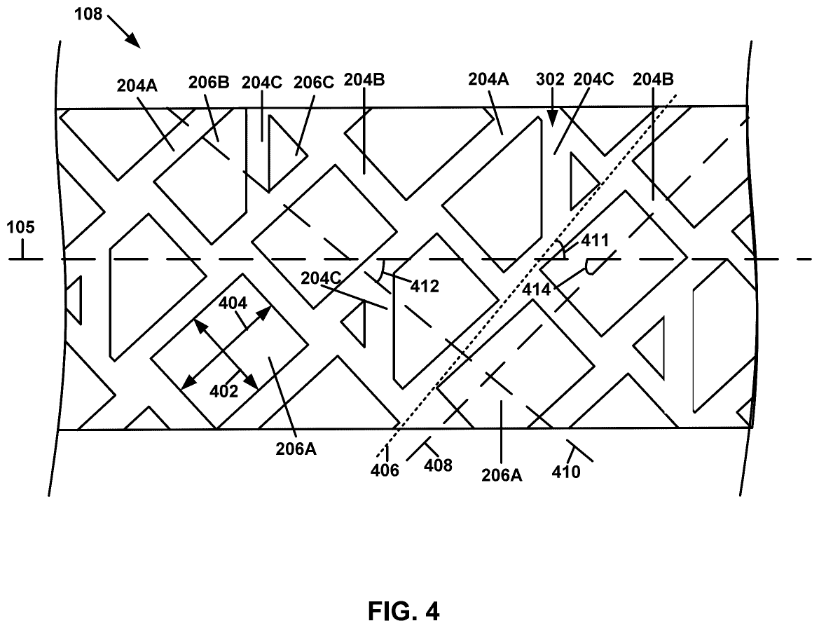

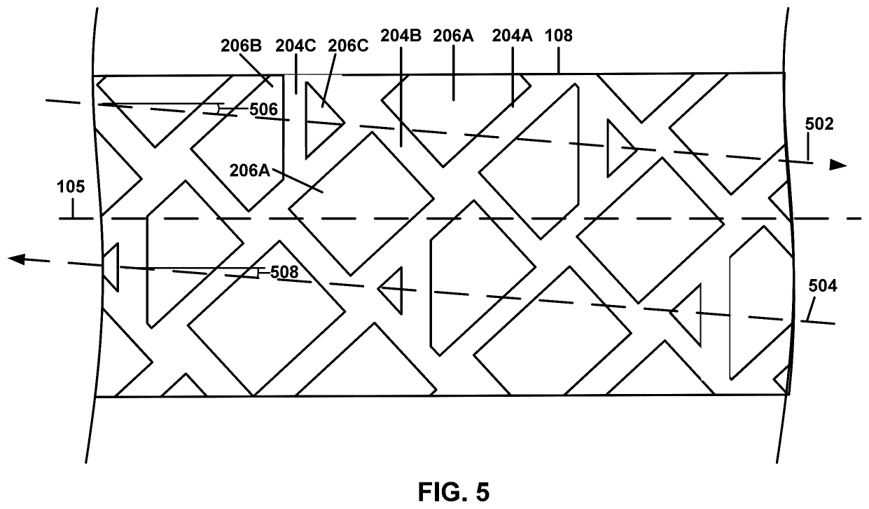

FIG. 4 is a perspective diagram illustrating a side view of a portion of the elongated body 108 of FIG. 1. FIG. 5 is a conceptual diagram illustrating another side view of a portion of the elongated body 108 of FIG. 1. As illustrated in FIGS. 4 and 5 and previously described herein, struts 204 may define openings 206 on outer surface 302 of elongated body 108. Struts 204 may include, but are not limited to, helical struts 204A, first cross struts 204B connecting at least two helical struts 204A, and second cross struts 204C connecting a helical strut 204A to a first cross strut 204B. Openings 206 may include a plurality of openings 206A, a plurality of openings 206B, and a plurality of openings 206C.

Helical struts 204A extend around the outer perimeter of elongated body 108 and along the longitudinal length of elongated body 108. Each helical strut 204A may define one or more coils 406 along the longitudinal length of elongated body 108. For each helical strut 204A, each coil 406 may correspond to a single complete revolution of helical strut 204A around the outer perimeter of elongated body 108. Coils 406 within each helical strut 204A may be separated by a pitch along longitudinal axis 105. The pitches between coils 406 may be uniform or may vary along the longitudinal length of elongated body 108. For example, coils 406 at distal portion 108A of elongated body 108 may define a tighter pitch than coils 406 at proximal portion 108B of elongated body 108. For each helical strut 406, coils 406 may define a helix extending around the outer perimeter of elongated body 108 and along the longitudinal length of elongated body 108. Coils 406 may extend along a reference plane offset from longitudinal axis by helix angle 411. Helix angle 411 may be uniform or may vary along the longitudinal length of elongated body 108.

Each helical strut 204A may define uniform or varying dimensions along the length of the helical strut 204A. For example, a helical strut 204A may be define a greater width 308 at one end of the helical strut 204A (e.g., at proximal portion 108B of elongated body 108) than a portion of helical strut 204A at an opposite end (e.g., at distal portion 108A of elongated body 108). In some examples, helical struts 204A may define stepwise changes in width along the length of helical struts 204A.

First cross struts 204B may connect two coils 406 of helical struts 204A. The two coils 406 may be longitudinally adjacent or may be longitudinally separated by one or more coils 406. In some examples, as illustrated in FIG. 4, first cross struts 204B may connect coils 406 from two separate helical struts 204A. In some examples, first cross struts 204B may connect coils 406 from a single helical strut 204A. First cross struts 204B may be perpendicular to coils 406 or may be offset from coils 406 by an angle. Second cross struts 204C may connect coils 406 of helical struts 204A to first cross struts 204B. Second cross struts 204C may be connected to first cross struts 204B and to helical struts 204A at locations along the length of struts 204A, 204B and between intersections between first cross struts 204B and helical struts 204A. In some examples, second cross struts 204C connect to first cross struts 204B and helical struts 204A at the intersections between first cross struts 204B and helical struts 204A. Each second cross strut 204C may connect a helical strut 204A to a first cross strut 204B that is the closest to the helical strut 204A (e.g., along longitudinal axis 105, around the outer perimeter of elongated body 108). In some examples, as illustrated in FIG. 4, each second cross strut 204C connects a first location along helical strut 204A to a second location along first cross strut 204B. The second location may correspond to a location on first cross struts 204B that shares a same longitudinal position as the first location and is the closest to the first location around the outer perimeter of elongated body 108. Second cross struts 204C may extend along a reference plane orthogonal to longitudinal axis 105. In some examples, the reference plane is offset from longitudinal axis by an angle less than helix angle 411.

Struts 204 may define dimensions that are same or similar to other struts 204. Struts 204 of the same type (e.g., helical struts 204A, first cross struts 204B, second cross struts 204C) may define uniform dimensions (e.g., depth 306 of sidewalls 307, widths 308A, 308B). In some examples, one or more struts 204 of the same type define different dimensions (e.g., at the same longitudinal locations along elongated body 108), than one or more other struts 204 of the same type. Struts 204 of different types may define the same, similar, or different dimensions. For example, struts 204A-C may define a uniform depth 306 and widths 308A, 308B.

Openings 206 may be defined by two or more struts 204 (e.g., by two or more of: at least one helical strut 204A, at least one first cross strut 204B, or at least one second cross strut 204C). Openings 206 may define a length 402 and a width 404. Lengths 402 and widths 404 may be uniform across openings 206 of a same type (e.g., openings 206A, openings 206B, openings 206C) or may vary along the longitudinal length of elongated body 108. Openings 206 may define triangular, rectangular, trapezoidal, quadrilateral, pentagonal, hexagonal, or other geometric shapes. The sides of openings 206 may be defined by sidewalls 307 of the struts 204 defining openings 206. Sidewalls 307 may extend from outer surface 302 of elongated body 108 towards inner surface 304 of elongated body 108. In some examples, openings 206 extend from outer surface 302 to a surface at a depth 306 from outer surface 302. In some examples, openings 206 extend from outer surface 302 through to inner lumen 305 of elongated body 108.

Openings 206 may define helical patterns 408, 410 around the outer perimeter of elongated body 108 and along the longitudinal length of elongated body 108. Helical patterns 408, 410 may each define less than one complete revolution, a single complete revolution, or more than one complete revolution around the outer perimeter of elongated body 108 along the longitudinal length of elongated body 108. Each of helical patterns 408, 410 may be defined by a corresponding helix angle. The helix angle may correspond to an offset angle from longitudinal axis 105 and may define the angle of one or more coils of openings 206 defining helical patterns 408, 410. The same openings 206 may define different helical patterns 408, 410 along different directions around longitudinal axis 105. For example, openings 206 may define a first helical pattern 408 with a first helix angle 412 in a first direction around longitudinal axis 105 and may define a second helical pattern 410 with a second helix angle 414 in a second direction around longitudinal axis 105. The first helix angle 412 may be the same as or may be different from the second helix angle 414. Helical patterns 408, 410 may inhibit preferential flexure of elongated body 108.

Openings 206A may each be defined by two first cross struts 204B connecting two coils 406 of helical struts 204A. Openings 206A may define a rectangular or quadrilateral shape (e.g., a parallelogram, a trapezoidal, or a rhombus shape). First cross struts 204B and helical struts 204A may form corners at the edges of openings 206A. The edges may be rounded or chamfered, e.g., to increase surface area between elongated body 108 and outer coating 210 and/or to reduce stress concentration on outer coating 210 at the edges.

Openings 206B may each be defined by one or more coils 406 of helical struts 204A, one first cross strut 204B, and one second cross strut 204C. Openings 206B may define a triangular or quadrilateral (e.g., trapezoidal) shape. Openings 206C may each be defined by one or more coils 406 of helical struts 204A, one first cross strut 204B, and one second cross strut 204C. Openings 206C may define a triangular or quadrilateral shape. Openings 206B may define a smaller, same, or larger surface area than openings 206C. Each opening 206B may be paired with a corresponding opening 206C. Each pair of openings 206B, 206C may be defined by a common second cross strut 204C. Each pair of openings 206B, 206C may be defined by the same coils 406 of helical struts 204A. Each opening of a pair of openings 206B, 206C is defined by a different first cross strut 204B.

In some examples, an opening 206A may be separated into a corresponding pair of openings 206B, 206C by second cross strut 204C. In some examples, a pair of openings 206B, 206C and the common second cross strut 204C may encompass a total surface area on the outer surface of elongated body 108 equal to an opening 206A. Elongated body 108 with second cross struts 204C and pairs of openings 206B, 206C may improve tensile stiffness, tensile strength, and/or torque responsiveness compared to an identical elongated body 108 without second cross struts 204C and pairs of openings 206B, 206C. Pairs of openings 206B, 206C may be separated along helical patterns 408, 410 by openings 206A. In some examples, pairs of openings 206B, 206C are helically alternating along a helical pattern (e.g., along helical pattern 410) or are helically separated by two or more openings 206A. In some examples, pairs of openings 206B, 206C are disposed on coils of openings of a helical pattern (e.g., helical pattern 408) and between longitudinally adjacent coils of openings 206A.

As illustrated in FIG. 5, pairs of openings 206B, 206C may define varying arrangements. Some of pairs of openings 206B, 206C may include openings 206B being proximal to openings 206C. Some of pairs of openings 206B, 206C may include openings 206C being proximal to openings 206B. Pairs of openings 206B, 206C may alternate arrangements along a helical pattern 410, e.g., to prevent elongated body 108 from exhibiting different tensile properties (e.g., tensile stiffness, tensile strength) in response to applications of external forces on elongated body 108 along longitudinal axis 105 and in different directions.

Pairs of openings 206B, 206C of similar arrangement may define helical patterns along the longitudinal length of elongated body 108 and around the outer perimeter of elongated body 108. For example, pairs of openings 206B, 206C of a first arrangement (e.g., opening 206C being proximal to opening 206B) may define one or more first helical patterns 502 along elongated body 108 and pairs of openings 206B, 206C of a second arrangement (e.g., openings 206C being distal to openings 206B) may define one or more helical patterns 502 along elongated body 108. Each helical pattern may define a corresponding helix angle offset from longitudinal axis 105 and defining the disposition of coil(s) of each helical pattern. For example, first helical pattern 502 may define a first helix angle 506 and second helical pattern may define a second helix angle 508. The helical patterns 502, 504 may cause an even distribution of tensile property values around the circumference of elongated body 108 and may cause elongated body 108 to respond to any tensile force in a same manner.

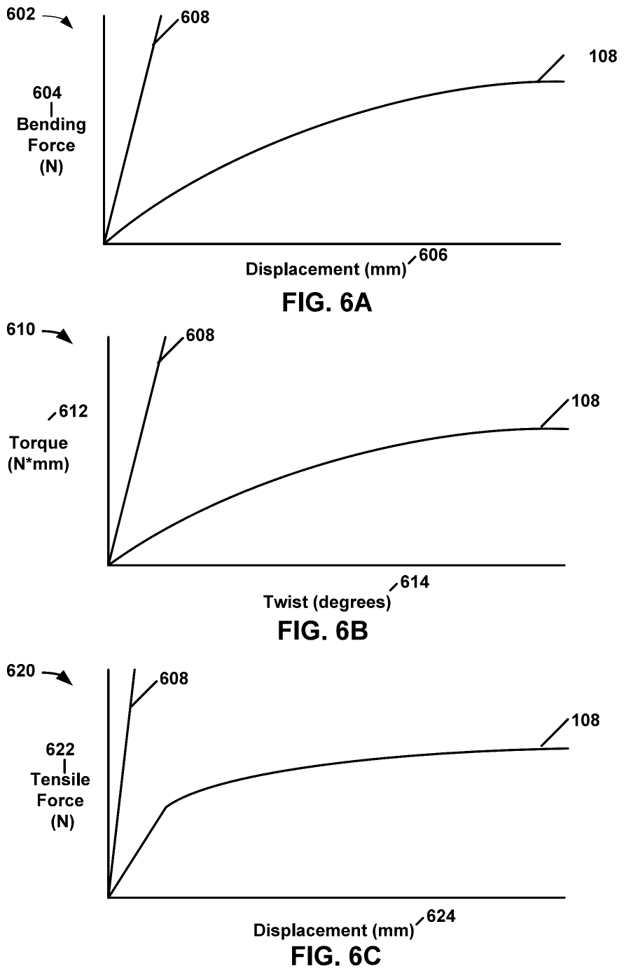

FIG. 6A is a graph 602 illustrating an example bending stiffness of the elongated body 108 of FIG. 1. Graph 602 illustrates bending forces 604 (e.g., in Newtons (N)) in response to displacement 606 (e.g., in millimeters (mm)) of elongated body 104. The displacement 606 may be along a reference plane orthogonal to longitudinal axis 105 of elongated body 104 and may be in response to a bending of elongated body 104, e.g., along the reference plane. Graph 602 illustrate example bending forces 604 on elongated body 108 and on an equivalent elongated body 608 in response to an amount of displacement 606 during a three-point bending flexural test (also referred to as a “three-point bending test”).

Equivalent elongated body 608 does not include any struts 204 or openings 206. In some examples, equivalent elongated body 608 is an elongated tube with an identical longitudinal length, outer diameter, inner lumen inner diameter, and thickness as elongated body 108. As illustrated in graph 602, elongated body 608 experiences greater forces 604 in response to a same amount of displacement 606 than elongated body 108. Struts 204 and openings 206 may reduce a bending stiffness of elongated body 108 to reduce an amount of force 604 experienced by elongated body 108 in response to displacement 606 as compared to elongated body 08. The reduced bending stiffness may increase the flexibility of elongated body 108 and may reduce a bending radius for elongated body 108. The increased flexibility and reduced bending radius may allow for the use of elongated body 108 in more tortuous portions of the vasculature of the patient and may provide the clinician with increase control in flexure of elongated body 108 as compared to elongated body 608.

FIG. 6B is a graph 610 illustrating an example torque responsiveness of the elongated body 108 of FIG. 1. Graph 610 illustrates torque 612 (e.g., in Newton-millimeters (N*mm)) experienced by elongated bodies 108, 608 in response to a degree of twist 614 of elongated bodies 108, 608. Graph 612 illustrates example torque 612 values in response to twisting of elongated bodies 108, 608 during a torsion test (e.g., a global torsion test or a global twist rotation test). During the torsion test, one end of an elongated body may be fixed, and a twisting force may be applied to an opposite end of the elongated body to measure the resulting torques 612 at various degrees of twist 614.

As illustrated in graph 610, elongated body 108 experiences reduced torque 612 for a same degree of twist 614 as compared to elongated body 608 and is less responsive to torque than elongated body 608. Struts 204 may increase torque 612 experienced by elongated body 108 in response to an applied torque than another elongated body with openings. Elongated body 108 may balance reduced torque responsiveness with increased flexibility (e.g., reduced bending stiffness) compared to elongated body 608 to improve performance and navigability of elongated body 108 within the patient compared to elongated body 608.

FIG. 6C is a graph 620 illustrating an example tensile stiffness of the elongated body 108 of FIG. 1. Graph 620 illustrates tensile forces 622 (e.g., in N) experienced by elongated bodies 108, 608 in response to tensile displacement 624 along longitudinal axes of elongated bodies 108, 608. Graph 620 illustrates example tensile force 622 values experienced by elongated bodies 108, 608 in response to a tensile pull test or a global tensile displacement test. During the test, one end of an elongated body may be fixed, and a pulling force may be applied to an opposite end of the elongated body to measure tensile force 622 results for varying levels of displacement 624.

As illustrated in graph 620, elongated body 108 may experience reduced tensile forces 622 than elongated body 608 for a same amount of tensile displacement 624. Elongated body 108 may exhibit reduced tensile stiffness and tensile strength compared to elongated body 608. Struts 204 and openings 206 may cause elongated body 108 to maintain or exhibit increased tensile stiffness and/or tensile strength compared to other elongated bodies with different openings.

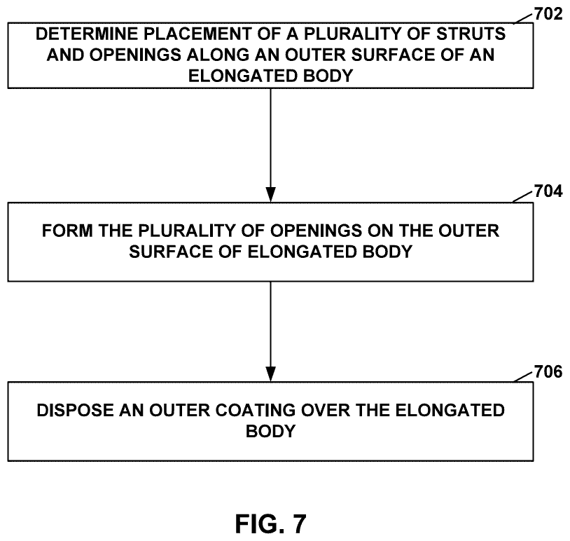

FIG. 7 is a flowchart illustrating an example process for forming the elongated body 108 of FIG. 1. While the example process illustrated in FIG. 7 is described herein primarily with respect to an elongated body 108 with struts 204 and openings 206 as described in FIGS. 2-5, the example process may be applied to elongated body 108 having any other struts or openings as described herein. FIG. 7 is described primarily with reference to a manufacturing system configured to form elongated body 108. The manufacturing system may include processing circuitry configured to retrieve and execute instructions to cause one or more other components of the manufacturing system (e.g., a cutting component) to perform one or more actions to form elongated body 108).

A manufacturing system may determine placement of a plurality of struts 204 and openings 206 along an outer surface 302 of an elongated body 108 of a medical device 102 (702). Elongated body 108 may define an elongated tube (e.g., a hypotube) or elongated annulus extending from a distal end 202A to a proximal end 202B along longitudinal axis 105 of device 102. Elongated body 108 may include an outer surface 302 and an inner surface 304. Inner surface 304 may define an inner lumen 305 extending along the longitudinal length of elongated body 108. Elongated body 108 may include struts 204 and openings 206 over at least a portion of outer surface 302 of elongated body 108. In some examples, struts 204 and openings 206 are disposed over an outer surface 302 of elongated body 108 outside of end sections 208 located at ends 202 of elongated body 108.

Each opening 206 may be defined by at least two struts 204. The at least two struts 204 may include at least two of: one or more helical struts 204A, one or more first cross struts 204B, or one or more second cross struts 204C. Each strut 204 may define uniform or varying dimensions (e.g., depths 306, widths 308A, 308B) along the length of the strut 204. Helical struts 204A may extend along the longitudinal length of elongated body 108. Helical struts 204A may extend around the outer perimeter to form one or more coils 406 forming a helix or spiral. First cross struts 204B may extend between coils 406 of helical struts 204A and second cross struts 204C may extend between a helical strut 204A and a first cross strut 204B. Struts 204 may define openings 206, e.g., sidewalls 307 of struts 204 may define the sides of openings 206. Openings 206 may extend from outer surface 302 of elongated body 108 towards inner surface 304 of elongated body 108. Openings 206 may extend from outer surface 302 through inner surface 304 to inner lumen 305 of elongated body 108.

The manufacturing system may determine the position and dimensions of struts 204 and openings 206 such that a completed elongated body 108 satisfies one or more threshold mechanical property values. The threshold mechanical properties may include, but are not limited to, a bending stiffness, a torque responsiveness, a tensile stiffness, or a tensile strength of elongated body 108. The manufacturing system may iteratively adjust the positioning and dimensions of struts 204 and openings 206 to optimize the mechanical properties of elongated body 108 to satisfy the threshold mechanical property values.

In some examples, the manufacturing system may adjust the dimensions and the positions of struts 204 and openings 206 until elongated body 108 exhibits a bending stiffness less than or equal to a maximum threshold bending stiffness, a torque responsiveness greater than or equal to a minimum threshold torque responsiveness, a tensile stiffness greater than or equal to a minimum threshold tensile stiffness, and/or a tensile strength greater than or equal to a minimum threshold tensile strength. The manufacturing system may determine the mechanical properties values for each iteration of the arrangement of struts 204 and openings 206 on elongated body 108 by applying one or more mechanical tests to elongated body 108 and compare the resulting mechanical property values to the threshold mechanical property values. The one or more mechanical tests may include, but are not limited to, a three-point bending test, a torsion test (e.g., a global torsion test or a global twist rotation test), or a tensile pull test (e.g., a global tensile pull test or a global tensile displacement test).

The manufacturing system may form the plurality of openings 206 on outer surface 302 of elongated body 108 (704). The manufacturing system may form struts 204 and openings 206 on elongated body 108 in accordance with the determined dimensions and placements of struts 204 and openings 206. The manufacturing system may control a cutting implement to remove material from elongated body 108 to form struts 204 and openings 206. The cutting implement may include, but are not limited to, a laser-cutting implement, an etching implement, or a mechanical cutting implement such as a blade, router, abrasion disk, or the like. The manufacturing system may form struts 204 via an additive manufacturing technique (e.g., a three-dimensional (3D) printing technique). For example, the manufacturing system may form second cross struts 204C in openings 206A to form openings 206B, 206C via an additive manufacturing technique.

The manufacturing system may dispose an outer coating 210 over elongated body 108 (706). Outer coating 210 may include one or more materials disposed over outer surface 302, within openings 206, and/or around struts 204 of elongated body 108. The one or more materials may be attached to outer surface 302 to sidewalls 207 of struts 204, and/or around struts 204 to affix outer coating 210 to elongated body 108 and inhibit unintended separation of outer coating 210 from elongated body 108. The one or more materials may include biocompatible polymers such as, but is not limited to, PTFE. The manufacturing system may heat the one or more materials up to a temperature of about 400° C. to flow the one or more materials across the outer surface 302 of elongated body 108, within openings 206, and/or around struts 204 of elongated body 108. A heat shrink material or heat shrink wrap may be disposed over the one or more materials of outer coating 210 to control the flow of the one or more materials and inhibit unintended flow of the one or more materials. The heat shrink material may facilitate control of the outer diameter and surface texture of device 102. Once the manufacturing system determines that the one or more materials is in contact with outer surfaces 302, sidewalls 207 of struts 204 forming openings 206, and/or around struts 204, the manufacturing system may remove the heat shrink material from outer coating 210 to allow outer coating 210 to adhere to elongated body 108.

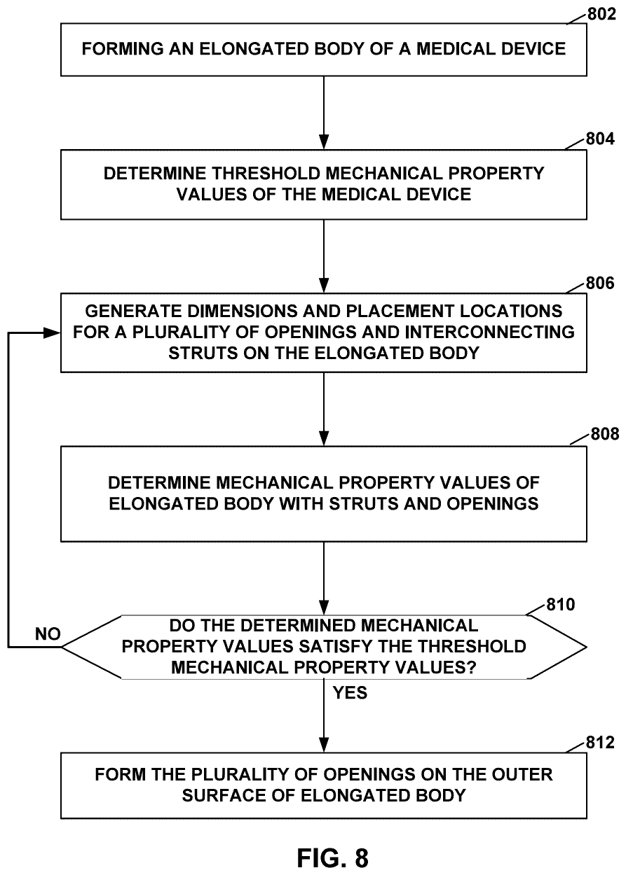

FIG. 8 is a flowchart illustrating another example process for forming the elongated body of FIG. 1. While the example process illustrated in FIG. 8 is described herein primarily with respect to an elongated body 108 with struts 204 and openings 206 as described in FIGS. 2-5, the example process may be applied to elongated body 108 having any other struts or openings as described herein. The manufacturing system may include processing circuitry configured to retrieve and execute instructions to simulate an elongated body 108 with struts 204 and openings 206 with different dimensions and placements and to cause one or more other components of the manufacturing system (e.g., a cutting component) to perform one or more actions to form elongated body 108.

A manufacturing system may form an elongated body 108 of a medical device 102 (802). Medical device 102 may include, but is not limited to, a medical catheter, a delivery catheter, or a guidewire. Medical device 102 may be configured to be navigated within one or more body lumen of the patient. The manufacturing system may form elongated body 108 by shaping a material into an elongated tube or elongated annulus. The material may include a biocompatible polymer or a biocompatible metallic alloy (e.g., nitinol). In some examples, the manufacturing system wraps the material around a mandrel to form the elongated tube or elongated annulus. The manufacturing system may shape the material into the elongated body 108 with specific outer diameter, length, and thickness values (e.g., from an outer surface 302 to inner surface 304). In some examples, the manufacturing system removes material from a pre-formed elongated tube or elongated annulus to form elongated body 108 with specific outer diameter, length, and thickness values. The manufacturing system may remove material via etching, laser-cutting, or mechanically cutting techniques.

The manufacturing system may determine threshold mechanical property values for medical device 102 (804). The mechanical properties of medical device 102 may include bending stiffness, torque responsiveness, tensile stiffness, and/or tensile strength. The threshold values may correspond to maximum or minimum values for the mechanical properties to allow a clinician to navigate medical device 102 within a body lumen of the patient to a target location within the patient. The threshold values may depend on the dimensions of medical device 102, the size (e.g., inner diameter) and tortuosity of the body lumen, and the target location. The manufacturing system may balance flexibility against pushability and torque responsiveness of medical device 102 to increase controllability of medical device 102 by the clinician.

The manufacturing system may generate dimensions and placement locations for a plurality of openings 206 and interconnecting struts 204 on elongated body 108 (806). The dimensions and placement locations for struts 204 and openings 206 may be selected to satisfy the threshold mechanical property values and to optimize controllability of medical device 102. The dimensions and placement locations for struts 204 may include, but are not limited to, types of struts 204 (e.g., helical struts 204A, first cross struts 204B, second cross struts 204C) depths 306 of struts 204, widths 308A, 308B of struts, length of struts 204, or a number and/or presence of any of helical struts 204A-C, The dimensions and placement locations for openings 206 may include, but are not limited to, types of openings 206 (e.g., openings 206A-C), a number of each type of openings 206, shapes of openings 206, depths of openings 206, lengths 402 and/or widths 404 of openings 206, helical patterns (e.g., helical patterns 408, 410, 502, 504) formed by openings 206, and/or helix angles (e.g., helix angles 412, 414, 506, 508) defining helical patterns. The manufacturing system may apply one or more finite element analysis (FEA) techniques to generate the dimensions and placement locations of struts 204 and openings 206. The FEA techniques may include, but are not limited to, an optimization algorithm such as TOSCA Optimization Technology available from Dassault Systèmes S.E., Vélizy-Villacoublay, France. In some examples, the optimization algorithm may include a non-parameterized optimization algorithm configured to improve mechanical properties related to navigability of the medical device, such as particular values or ranges of values for tensile strength, tensile stiffness, bending stiffness, or the like.

The manufacturing system may apply the determined mechanical property values of elongated body 108 with struts 204 and openings 206 (808). Struts 204 and openings 206 may be disposed on outer surface 302 of elongated body 108 in accordance with the generated dimensions and placement locations. The manufacturing system may apply or simulate one or more mechanical tests to elongated body 108 to determine the mechanical property values of elongated body 108. The mechanical tests may include, but are not limited to, a three-point bending test, a torsion test (e.g., a global torsion test or a global twist rotation test), or a tensile pull test (e.g., a global tensile pull test or a global tensile displacement test). The manufacturing system may determine localized or global values for the mechanical properties based on the mechanical tests in response to varying levels of bending forces, torque, or tensile forces applied to elongated body 108. The manufacturing system may determine levels of bending forces 604, torque 612, or tensile forces 622 corresponding to different amounts of displacement by elongated body 108 (e.g., bending displacement 606, tensile displacement 624) or to different degrees of twist 614 of elongated body 108.

The manufacturing system may compare the determined mechanical property values to the threshold mechanical property values (810). Based on a determination that the determined mechanical property values satisfy the threshold mechanical property values (“YES” branch of 810), the manufacturing system may form the plurality of openings 206 on outer surface 302 of elongated body 108 (812). The manufacturing system may form struts 204 and openings 206 on elongated body 108, e.g., as previously described in FIG. 7.

Based on a determination that the determined mechanical property values do not satisfy the threshold mechanical property values, (“NO” branch of 810), the manufacturing system may generate new dimensions and placement locations for openings 206 and struts 204 on elongated body 108 (806). The manufacturing system may iteratively adjust dimensions and placement locations for openings 206 and struts 204 on elongated body 108 until the manufacturing system determines that elongated body 108 satisfies the threshold mechanical property values (“YES” branch of 810).

The examples described herein may be combined in any permutation or combination. The disclosure herein describes all of the following examples.
Example 1: a medical device comprising: an elongated body extending along a longitudinal axis from a proximal end to a distal end, wherein the elongated body comprises: an inner surface defining an inner lumen extending along the longitudinal axis, and a plurality of interconnecting struts extending around an outer perimeter of the elongated body and defining a plurality of openings disposed on a portion of an outer surface of the elongated body and along the longitudinal axis and extending towards the inner lumen of the elongated body, wherein the plurality of interconnecting struts comprises: a plurality of helical struts, wherein each helical strut of the plurality of helical struts extends along the elongated body from the proximal end to the distal end and around the outer perimeter of the elongated body, and wherein the plurality of helical struts defines one or more coils extending around the outer perimeter along a longitudinal length of the elongated body, a plurality of first cross struts, wherein each first cross strut extends between longitudinally adjacent coils of the one or more coils, and a plurality of second cross struts, wherein each second cross strut extends between a first cross strut of the plurality of first cross struts and a coil of a helical strut of the plurality of helical struts, wherein each opening is defined by two or more of: one or more helical struts of the plurality of helical struts, one or more first cross struts of the plurality of first cross struts, or one or more second cross struts of the plurality of second cross struts; and an outer coating disposed on the outer surface of the elongated body, wherein at least a portion of the outer coating is disposed on sidewalls of the plurality of interconnecting struts.
Example 2: the medical device of example 1, wherein the plurality of openings defines a helical pattern wound around the longitudinal axis and extending along the longitudinal length of the elongated body.
Example 3: the medical device of any of examples 1 and 2, wherein one or more openings of the plurality of openings extends between the outer surface and the inner surface of the elongated body.
Example 4: the medical device of any of examples 1-3, wherein the plurality of openings comprises: a plurality of first openings, wherein each first opening is defined by one or more helical struts and one or more first cross struts, a plurality of second openings, wherein each second opening is defined by one or more helical struts, one or more first cross struts, and one or more second cross struts, and a plurality of third openings, wherein each third opening is defined by one or more helical struts, one or more first cross struts, and one or more second cross struts, wherein a second opening is separated from a corresponding third opening by a second cross strut, wherein a combined area of the second opening, the third opening, and the second cross strut is equal to an area of a first opening, and wherein the second cross strut is configured to increase tensile strength of the elongated body.
Example 5: the medical device of example 4, wherein each first opening of the plurality of first openings defines a rectangular shape, wherein each second opening of the plurality of second openings defines a first quadrilateral or a first triangular shape, and wherein each third opening of the plurality of third openings defines a second quadrilateral or a second triangular shape, wherein the first quadrilateral shape is different from the second quadrilateral shape, and wherein the first triangular shape is different from the second triangular shape.
Example 6: the medical device of any of examples 4 and 5, wherein the second opening, the third opening, and the second cross strut are disposed between two helically adjacent first openings.
Example 7: the medical device of any of examples 1-6, wherein the plurality of openings defines a first helix wound around the longitudinal axis of the elongated body in a first direction and a second helix wound around the longitudinal axis of the elongated body in a second direction, and wherein the first direction is different from the second direction.
Example 8: the medical device of example 7, wherein the first helix and the second helix intersect at one or more locations along the longitudinal length of the elongated body.
Example 9: the medical device of any of examples 1-8, wherein the outer coating is configured to reflow around at least one interconnecting strut of the plurality of interconnecting struts in response to heating to a temperature of up to about 400° C.
Example 10: the medical device of any of examples 1-9, wherein the elongated body comprises a hypotube comprising a metallic alloy.
Example 11: the medical device of example 10, wherein each opening of the plurality of openings is formed via a laser-cutting technique.
Example 12: a method of manufacturing a medical device, the method comprising: receiving, by processing circuitry of a device manufacturing system, a set of dimensions and locations of a plurality of openings disposed on an elongated body of the medical device based on mechanical properties of the medical device, wherein the elongated body comprises: an outer surface, an inner surface defining an inner lumen extending along a longitudinal axis, and a plurality of interconnecting struts extending around an outer perimeter of the elongated body and defining the plurality of openings extending from the outer surface towards the inner surface, wherein the plurality of interconnecting struts comprises: a plurality of helical struts, wherein each helical strut of the plurality of helical struts extends along the elongated body from the proximal end to the distal end and around the outer perimeter of the elongated body, and wherein the plurality of helical struts defines one or more coils extending around the outer perimeter along a longitudinal length of the elongated body, a plurality of first cross struts, wherein each first cross strut extends between longitudinally adjacent coils of the one or more coils, and a plurality of second cross struts, wherein each second cross strut extends between a first cross strut of the plurality of first cross struts and a coil of a helical strut of the plurality of helical struts, wherein each opening is defined by two or more of: one or more helical struts of the plurality of helical struts, one or more first cross struts of the plurality of first cross struts, or one or more second cross struts of the plurality of second cross struts; and causing, by the processing circuitry and based on the set of dimensions and locations of the plurality of openings, a cutting component of the device manufacturing system to form the plurality of openings on the outer surface of the portion of the elongated body.
Example 13: the method of example 12, wherein the plurality of openings defines a helical pattern wound around the longitudinal axis and extending along the longitudinal length of the elongated body.
Example 14: the method of any of examples 12 and 13, wherein one or more openings of the plurality of openings extends between the outer surface and the inner surface of the elongated body.
Example 15: the method of any of examples 12-14, further comprising: forming an outer layer around the outer surface of the elongated body by at least: disposing a material defining the outer layer into the plurality of openings, and affixing the material on sidewalls of the plurality of interconnecting struts.
Example 16: the method of any of examples 12-15, further comprising: determining, by the processing circuitry, the mechanical properties of the elongated body of the medical device; and determining, by the processing circuitry, the set of dimensions and locations of the plurality of openings to satisfy threshold conditions of mechanical properties of the elongated body.
Example 17: the method of any of examples 12-16, wherein the plurality of openings comprises: a plurality of first openings, wherein each first opening is defined by one or more helical struts and one or more first cross struts, a plurality of second openings, wherein each second opening is defined by one or more helical struts, one or more first cross struts, and one or more second cross struts, and a plurality of third openings, wherein each third opening is defined by one or more helical struts, one or more first cross struts, and one or more second cross struts, wherein a second opening corresponds to a third opening and is separated from the corresponding third opening by a second cross strut, wherein a combined area of the second opening, the third opening, and the second cross strut is equal to an area of a first opening, and wherein the second cross strut is configured to increase tensile strength of the elongated body.
Example 18: the method of example 17, wherein each first opening of the plurality of first openings defines a rectangular shape, wherein each second opening of the plurality of second openings defines a first quadrilateral or a first triangular shape, and wherein each third opening of the plurality of third openings defines a second quadrilateral or a second triangular shape, wherein the first quadrilateral shape is different from the second quadrilateral shape, and wherein the first triangular shape is different from the second triangular shape.
Example 19: the method of any of examples 12-18, wherein the second opening, the third opening, and the second connecting strut are disposed between two helically adjacent first openings.
Example 20: the method of any of examples 12-19, wherein the mechanical properties comprise one or more of a maximum bending force of the catheter, a reaction movement to twisting of the catheter, or a tensile strength of the catheter.
Example 21: the method of example 20, wherein the maximum bending force is determined by a three-point bending displacement test, wherein the reaction movement to twisting is determined by a global twist rotation test, and wherein the tensile strength is determined by a global tensile displacement test.
Example 22: the method of any of examples 12-21, further comprising determining the mechanical properties of the medical device by at least: determining, by the processing circuitry, threshold mechanical property values of the medical device, generating, by the processing circuitry, a set of dimensions and locations of the plurality of openings; determining, by the processing circuitry, mechanical property values of the medical device having the plurality of openings defining by the generated set of dimensions and locations, comparing, by the processing circuitry, the determined mechanical property values to the threshold mechanical property values, and in response to determining that the determined mechanical properties values satisfy the threshold mechanical property values, transmitting, by the processing circuitry, cutting instructions to the cutting component, the cutting instructions being configured to cause the cutting component to form the plurality of openings on the elongated body based on the generated set of dimensions and locations of the plurality of openings.
Example 23: the method of any of examples 12-22, wherein the elongated body comprises a hypotube comprising a metallic alloy.
Example 24: the method of any of examples 12-23, wherein the cutting component comprises a laser-cutting device.
Example 25: the method of any of examples 12-24, wherein each opening of the plurality of openings define a first set of dimensions at a first location and a second set of dimensions at a second location, wherein the first location is disposed on the outer surface of the elongated body, wherein the second location is radially inwards of the first location, and wherein the first set of dimensions define different dimension values than the second set of dimensions.
Example 26: a system comprising: a catheter comprising: an elongated body extending along a longitudinal axis from a proximal end to a distal end, wherein the elongated body comprises: an inner surface defining an inner lumen extending along the longitudinal axis, and a plurality of interconnecting struts extending around an outer perimeter of the elongated body and defining a plurality of openings disposed on a portion of an outer surface of the elongated body and along the longitudinal axis, wherein each opening of the plurality of openings extends from the outer surface towards the inner surface, wherein the plurality of interconnecting struts comprises: a plurality of helical struts, wherein each helical strut of the plurality of helical struts extends along the elongate body from the proximal end to the distal end and around an outer perimeter of the elongated body, and wherein the plurality of helical struts defines one or more coils extending around the outer perimeter along a longitudinal length of the elongated body, a plurality of first cross struts, wherein each first cross strut extends between longitudinally adjacent coils of the one or more coils, and a plurality second cross struts, wherein each second cross strut extends between a first cross strut of the plurality of first cross struts and a coil of a helical strut of the plurality of helical struts, wherein each opening is defined by two or more of: one or more helical struts of the plurality of helical struts, one or more first cross struts of the plurality of first cross struts, or one or more second cross struts of the plurality of second cross struts; and an outer coating disposed on the outer surface of the elongated body, wherein at least a portion of the outer coating is disposed on sidewalls of the plurality of struts; and a guidewire configured to be navigated within the inner lumen of the elongated body of the catheter.
Example 27: the system of example 26, wherein the plurality of openings defines a helical pattern wound around the longitudinal axis and extending along the longitudinal length of the elongated body.
Example 28: the system of any of examples 26 and 27, wherein one or more openings of the plurality of openings extends between the outer surface and the inner surface of the elongated body.
Example 29: the system of any of examples 26-28, wherein the plurality of openings comprises: a first plurality of first openings and a second plurality of openings, wherein each opening of the first plurality of openings defines a first geometric shape, wherein each first opening is defined by one or more helical struts and one or more first cross struts, a plurality of second openings, wherein each second opening is defined by one or more helical struts, one or more first cross struts, and one or more second cross struts, and a plurality of third openings, wherein each third opening is defined by one or more helical struts, one or more first cross struts, and one or more second cross struts, wherein a second opening corresponds to a third opening and is separated from the corresponding third opening by a second cross strut, wherein a combined area of the second opening, the third opening, and the second cross strut is equal to an area of a first opening, and wherein the second cross strut is configured to increase tensile strength of the elongated body.
Example 30: the system of example 29, wherein each first opening of the plurality of first openings defines a rectangular shape, wherein each second opening of the plurality of second openings defines a first quadrilateral or a first triangular shape, and wherein each third opening of the plurality of third openings defines a second quadrilateral or a second triangular shape, wherein the first quadrilateral shape is different from the second quadrilateral shape, and wherein the first triangular shape is different from the second triangular shape.
Example 31: the system of any of examples 29 and 30, wherein the second opening, the third opening, and the second connecting strut are disposed between two helically adjacent first openings.
Example 32: the system of any of examples 26-31, wherein the plurality of openings define a first helix wound around the longitudinal axis of the elongated body in a first direction and a second helix wound around the longitudinal axis of the elongated body in a second direction, and wherein the first direction is different from the second direction.
Example 33: the system of example 32, wherein the first helix and the second helix intersect at one or more locations along the longitudinal length of the elongated body.
Example 34: the system of any of examples 26-33, wherein the outer coating is configured to reflow around at least one interconnecting strut of the plurality of interconnecting struts in response to heating to a temperature of up to about 400° C.
Example 35: the system of any of examples 26-34, wherein the elongated body comprises a hypotube comprising a metallic alloy.
Example 36: the system of example 35, wherein each opening of the plurality of openings is formed via a laser-cutting technique.

Various aspects of the disclosure have been described. These and other aspects are within the scope of the following claims.

What is claimed is:

1. A medical device comprising:
an elongated body extending along a longitudinal axis from a proximal end to a distal end, wherein the elongated body comprises:
an inner surface defining an inner lumen extending along the longitudinal axis, and
a plurality of interconnecting struts extending around an outer perimeter of the elongated body and defining a plurality of openings disposed on a portion of an outer surface of the elongated body and along the longitudinal axis and extending towards the inner lumen of the elongated body, wherein the plurality of interconnecting struts comprises:
a plurality of helical struts, wherein each helical strut of the plurality of helical struts extends along the elongated body from the proximal end to the distal end and around the outer perimeter of the elongated body, and wherein the plurality of helical struts defines one or more coils extending around the outer perimeter along a longitudinal length of the elongated body,
a plurality of first cross struts, wherein each first cross strut extends between longitudinally adjacent coils of the one or more coils, and
a plurality of second cross struts, wherein each second cross strut extends between a first cross strut of the plurality of first cross struts and a coil of a helical strut of the plurality of helical struts,
wherein each opening is defined by two or more of:
one or more helical struts of the plurality of helical struts,
one or more first cross struts of the plurality of first cross struts, or
one or more second cross struts of the plurality of second cross struts; and
an outer coating disposed on the outer surface of the elongated body, wherein at least a portion of the outer coating is disposed on sidewalls of the plurality of interconnecting struts.

2. The medical device of claim 1, wherein the plurality of openings defines a helical pattern wound around the longitudinal axis and extending along the longitudinal length of the elongated body, and wherein one or more openings of the plurality of openings extends between the outer surface and the inner surface of the elongated body.

3. The medical device of claim 1, wherein the plurality of openings comprises:
a plurality of first openings, wherein each first opening is defined by one or more helical struts and one or more first cross struts,
a plurality of second openings, wherein each second opening is defined by one or more helical struts, one or more first cross struts, and one or more second cross struts, and
a plurality of third openings, wherein each third opening is defined by one or more helical struts, one or more first cross struts, and one or more second cross struts,
wherein a second opening is separated from a corresponding third opening by a second cross strut,
wherein a combined area of the second opening, the third opening, and the second cross strut is equal to an area of a first opening, and
wherein the second cross strut is configured to increase tensile strength of the elongated body.

4. The medical device of claim 3,
wherein each first opening of the plurality of first openings defines a rectangular shape,
wherein each second opening of the plurality of second openings defines a first quadrilateral or a first triangular shape, and
wherein each third opening of the plurality of third openings defines a second quadrilateral or a second triangular shape,
wherein the first quadrilateral shape is different from the second quadrilateral shape, and wherein the first triangular shape is different from the second triangular shape.

5. The medical device of any of claim 4, wherein the second opening, the third opening, and the second cross strut are disposed between two helically adjacent first openings.

6. The medical device of claim 1, wherein the plurality of openings defines a first helix wound around the longitudinal axis of the elongated body in a first direction and a second helix wound around the longitudinal axis of the elongated body in a second direction, and wherein the first direction is different from the second direction, and
wherein the first helix and the second helix intersect at one or more locations along the longitudinal length of the elongated body.

7. The medical device of claim 1, wherein the outer coating is configured to reflow around at least one interconnecting strut of the plurality of interconnecting struts in response to heating to a temperature of up to about 400° C.

8. The medical device of claim 1, wherein the elongated body comprises a hypotube comprising a metallic alloy, and
wherein each opening of the plurality of openings is formed via a laser-cutting technique.

9. A method of manufacturing the medical device of claim 1, the method comprising:
receiving, by processing circuitry of a device manufacturing system, a set of dimensions and locations of the plurality of openings disposed on the elongated body of the medical device based on mechanical properties of the medical device,
and
causing, by the processing circuitry and based on the set of dimensions and locations of the plurality of openings, a cutting component of the device manufacturing system to form the plurality of openings on the outer surface of the portion of the elongated body.

10. The method of claim 9, wherein the plurality of openings defines a helical pattern wound around the longitudinal axis and extending along the longitudinal length of the elongated body.

11. The method of claim 9, wherein one or more openings of the plurality of openings extends between the outer surface and the inner surface of the elongated body.

12. The method of any claim 9, further comprising:
forming an outer layer around the outer surface of the elongated body by at least:
disposing a material defining the outer layer into the plurality of openings, and
affixing the material on sidewalls of the plurality of interconnecting struts.

13. The method of claim 9, further comprising:
determining, by the processing circuitry, the mechanical properties of the elongated body of the medical device; and
determining, by the processing circuitry, the set of dimensions and locations of the plurality of openings to satisfy threshold conditions of mechanical properties of the elongated body.

14. The method of claim 9, wherein the plurality of openings comprises:
a plurality of first openings, wherein each first opening is defined by one or more helical struts and one or more first cross struts,
a plurality of second openings, wherein each second opening is defined by one or more helical struts, one or more first cross struts, and one or more second cross struts, and
a plurality of third openings, wherein each third opening is defined by one or more helical struts, one or more first cross struts, and one or more second cross struts,
wherein a second opening corresponds to a third opening and is separated from the corresponding third opening by a second cross strut, wherein a combined area of the second opening, the third opening, and the second cross strut is equal to an area of a first opening, and wherein the second cross strut is configured to increase tensile strength of the elongated body.

15. The method of claim 14,
wherein each first opening of the plurality of first openings defines a rectangular shape,
wherein each second opening of the plurality of second openings defines a first quadrilateral or a first triangular shape, and
wherein each third opening of the plurality of third openings defines a second quadrilateral or a second triangular shape,
wherein the first quadrilateral shape is different from the second quadrilateral shape, and wherein the first triangular shape is different from the second triangular shape, and wherein the second opening, the third opening, and the second connecting strut are disposed between two helically adjacent first openings.

16. The method of claim 9, wherein the mechanical properties comprise one or more of a maximum bending force of the catheter, a reaction movement to twisting of the catheter, or a tensile strength of the catheter, and
wherein the maximum bending force is determined by a three-point bending displacement test, wherein the reaction movement to twisting is determined by a global twist rotation test, and wherein the tensile strength is determined by a global tensile displacement test.

17. The method of claim 9, further comprising determining the mechanical properties of the medical device by at least:
determining, by the processing circuitry, threshold mechanical property values of the medical device,
generating, by the processing circuitry, a set of dimensions and locations of the plurality of openings;
determining, by the processing circuitry, mechanical property values of the medical device having the plurality of openings defining by the generated set of dimensions and locations,
comparing, by the processing circuitry, the determined mechanical property values to the threshold mechanical property values, and
in response to determining that the determined mechanical properties values satisfy the threshold mechanical property values, transmitting, by the processing circuitry, cutting instructions to the cutting component, the cutting instructions being configured to cause the cutting component to form the plurality of openings on the elongated body based on the generated set of dimensions and locations of the plurality of openings.

18. A system comprising:
a catheter comprising:
an elongated body extending along a longitudinal axis from a proximal end to a distal end, wherein the elongated body comprises:
an inner surface defining an inner lumen extending along the longitudinal axis, and
a plurality of interconnecting struts extending around an outer perimeter of the elongated body and defining a plurality of openings disposed on a portion of an outer surface of the elongated body and along the longitudinal axis, wherein each opening of the plurality of openings extends from the outer surface towards the inner surface, wherein the plurality of interconnecting struts comprises:
a plurality of helical struts, wherein each helical strut of the plurality of helical struts extends along the elongate body from the proximal end to the distal end and around an outer perimeter of the elongated body, and wherein the plurality of helical struts defines one or more coils extending around the outer perimeter along a longitudinal length of the elongated body,
a plurality of first cross struts, wherein each first cross strut extends between longitudinally adjacent coils of the one or more coils, and
a plurality second cross struts, wherein each second cross strut extends between a first cross strut of the plurality of first cross struts and a coil of a helical strut of the plurality of helical struts,
wherein each opening is defined by two or more of:
one or more helical struts of the plurality of helical struts,
one or more first cross struts of the plurality of first cross struts, or
one or more second cross struts of the plurality of second cross struts; and
an outer coating disposed on the outer surface of the elongated body, wherein
at least a portion of the outer coating is disposed on sidewalls of the plurality of struts; and
a guidewire configured to be navigated within the inner lumen of the elongated body of the catheter, and
wherein the plurality of openings defines a helical pattern wound around the longitudinal axis and extending along the longitudinal length of the elongated body.

19. The system of claim 18, wherein the plurality of openings comprises:
a first plurality of first openings and a second plurality of openings, wherein each opening of the first plurality of openings defines a first geometric shape, wherein each first opening is defined by one or more helical struts and one or more first cross struts,
a plurality of second openings, wherein each second opening is defined by one or more helical struts, one or more first cross struts, and one or more second cross struts, and
a plurality of third openings, wherein each third opening is defined by one or more helical struts, one or more first cross struts, and one or more second cross struts,
wherein a second opening corresponds to a third opening and is separated from the corresponding third opening by a second cross strut,
wherein a combined area of the second opening, the third opening, and the second cross strut is equal to an area of a first opening, and
wherein the second cross strut is configured to increase tensile strength of the elongated body.

20. The system of claim 19,
wherein each first opening of the plurality of first openings defines a rectangular shape,
wherein each second opening of the plurality of second openings defines a first quadrilateral or a first triangular shape,
wherein each third opening of the plurality of third openings defines a second quadrilateral or a second triangular shape,
wherein the first quadrilateral shape is different from the second quadrilateral shape, and wherein the first triangular shape is different from the second triangular shape, wherein the plurality of openings define a first helix wound around the longitudinal axis of the elongated body in a first direction and a second helix wound around the longitudinal axis of the elongated body in a second direction, and wherein the first direction is different from the second direction, and
wherein the first helix and the second helix intersect at one or more locations along the longitudinal length of the elongated body.
